(19) United States
(12) Patent Application Publication
(10) Pub. No.: US 2026/0272301 A1
(43) Pub. Date: Sep. 17, 2026

(54) METHOD FOR PROVIDING ALIGNMENT SCAN PARAMETERS FOR A MAGNETIC RESONANCE APPARATUS

(71) Applicant: Siemens Healthineers AG, Forchheim (DE)

(72) Inventors: Dominik PAUL, Bubenreuth (DE); Fabian WAGNER, Erlangen (DE); Maria Jimena COSTA, Nuernberg (DE)

(73) Assignee: Siemens Healthineers AG, Forchheim (DE)

(21) Appl. No.: 19/567,616

(22) Filed: Mar. 16, 2026

(30) Foreign Application Priority Data
Mar. 17, 2025 (DE) 10 2025 110 083.7

(51) Int. Cl. A61B 5/00; A61B 5/055; G01R 33/54; G01R 33/58

(52) U.S. Cl. CPC A61B 5/0037; A61B 5/055; G01R 33/543; G01R 33/58

(57) ABSTRACT

A method for providing alignment scan parameters for an alignment scan of a magnetic resonance examination of a patient's body part via a magnetic resonance apparatus comprises: capturing a normalization scan, wherein the normalization scan is used to calibrate the magnetic resonance apparatus; determining the position of the body part based on the normalization scan; positioning a local coil of the magnetic resonance apparatus in a patient-receiving region of the magnetic resonance apparatus around the body part; ascertaining alignment scan parameters for an alignment scan based on the position of the body part; and providing the alignment scan parameters.

### CROSS-REFERENCE TO RELATED APPLICATION(S)

The present application claims priority under 35 U.S.C. § 119 to German Patent Application No. 10 2025 110 083.7, filed Mar. 17, 2025, the entire contents of which is incorporated herein by reference.

### FIELD

One or more example embodiments of the present invention relate to an alignment scan of a magnetic resonance examination via a magnetic resonance unit. In particular, one or more example embodiments of the present invention relate to positioning and/or position detection of a local coil on a patient's body part for a magnetic resonance examination via a magnetic resonance unit.

### BACKGROUND

Medical imaging, in particular magnetic resonance imaging (MRI), is a well-known diagnostic tool for examining patients' body parts, such as the extremities of the human body. However, a common problem in imaging body parts, and in particular extremities, is confusion between the sides. In particular, this can occur with body parts that typically occur in plurality and on both the left and right sides of the human body. This can result in the incorrect anatomy being displayed. Such confusion can, for example, necessitate repeat imaging. Repeat imaging in turn can result in losses in time and money.

In addition to incorrect and/or unknown positioning of the local coil on the patient and/or within the magnetic resonance apparatus, inconsistencies can occur in automated examination procedures. Protocols for magnetic resonance examinations are, for example, automatically adapted and/or positioned with respect to the patient's body part to be examined (also referred to as auto-align). Typically, an overview image (also known as an alignment scan) is acquired as a basis for automatic positioning and used for landmark detection of the body region. A magnetic resonance scan can be planned based on the alignment scan.

It is known to acquire an alignment scan covering the entire patient-receiving region. Herein, for example, a magnetic resonance scan of a wrist requires an alignment scan comprising the position of the hand to the right of the body, the position of the hand to the left of the body and the hand raised above the head (so-called superman position). In addition, the required resolution of the alignment scan depends on the body region to be examined. For example, a high-resolution alignment scan is required for a wrist. This results in time-consuming and expensive alignment scans.

### SUMMARY

One or more example embodiments of the present invention are based on an object of improving the efficiency of magnetic resonance examinations, in particular alignment scans. In particular, an object of one or more example embodiments of the present invention can be considered to be the determination of an optimized acquisition region for an alignment scan for a magnetic resonance examination.

According to one or more example embodiments of the present invention, a method is proposed for providing alignment scan parameters for an alignment scan of a magnetic resonance examination of a patient's body part via a magnetic resonance apparatus. The method comprises capturing a normalization scan. The normalization scan is used to calibrate the magnetic resonance apparatus. The method comprises determining the position of the body part based on the normalization scan. The local coil is positioned in a patient-receiving region of the magnetic resonance apparatus around the patient's body part. The method comprises ascertaining alignment scan parameters for an alignment scan based on the determined position of the body part. The method comprises providing the alignment scan parameters.

The normalization scan is in particular used to calibrate the local coil. In particular, the normalization scan is used to calibrate the signal parameters of a local coil of the magnetic resonance apparatus.

The magnetic resonance apparatus is typically embodied to capture medical and/or diagnostic image data from a patient. In particular, the magnetic resonance apparatus can be embodied to acquire medical image data from a predetermined body region of a patient via one and/or more local coils. The magnetic resonance apparatus can comprise one and/or more local coils. The magnetic resonance apparatus can be connected to one and/or more local coils for signal transmission.

Furthermore, the magnetic resonance apparatus typically comprises a magnet unit, a patient table and a control unit. The magnet unit surrounds a patient-receiving region bounded by an inner wall of the magnet unit and comprises a whole-body coil. The patient table can be moved into the patient-receiving region. The magnet bore can define and/or comprise the patient-receiving region. In particular, the patient's body part is positioned in the magnet bore from which imaging and/or spectroscopic magnetic resonance signals are to be captured by the magnetic resonance apparatus. In particular, at least part of the patient is positioned in an isocenter of the magnetic resonance apparatus, in particular of a main magnetic field. The irradiation of the radio-frequency transmission pulses typically generates radio-frequency fields during the magnetic resonance examination and these deflect nuclear spins in the patient from their rest position. Subsequent relaxation generates imaging and/or spectroscopic magnetic resonance signals, which are received by one or more receiving antennas of the magnetic resonance apparatus and can be used for the reconstruction of magnetic resonance images or for spectroscopy. In particular, the whole-body coil, which is permanently integrated into a magnet unit of the magnetic resonance apparatus, comprises at least one receiving antenna and/or one transmitting antenna.

While the whole-body coil is in particular embodied to acquire large areas of a patient, a local coil can be used as an additional receiver and/or detector in order to capture image data from specific body parts. The local coil is embodied to be placed directly on and/or in the immediate vicinity of the body part to be examined in order to receive signals from the tissue of the body part via an integrated receiving antenna. In particular, a local coil can comprise a radio-frequency receiving antenna. Various types of local coils are known for different body parts, for example orthopedic coils for examining joints, such as knees, shoulders, wrists or ankles. The shape and dimensions of local coils are typically adapted to the respective anatomy of the body part to be examined. In addition, flexible coils for use on different body parts are known.

In particular, a magnetic resonance scan of the patient's body part can be captured using the magnetic resonance apparatus. Capturing a magnetic resonance scan can comprise performing an imaging examination using the magnetic resonance apparatus based on an imaging protocol. During the imaging examination, also referred to as a magnetic resonance examination and/or magnetic resonance scan, the patient is typically located wholly or partially in the patient-receiving region. In particular, the patient can be positioned on a patient support surface of the patient table during the imaging examination. To generate imaging and/or spectroscopic magnetic resonance signals during an imaging examination, radio-frequency transmission pulses are irradiated into the patient-receiving region, in particular into the patient, in particular into a body part, using one or more transmitting antennas in accordance with a measurement protocol, in particular a magnetic resonance sequence. The radio-frequency transmission pulses can be captured via one or more receiving antennas.

“Body part” refers to a partial area and/or partial region of a patient's body. In the context of embodiments of the present invention, the term “body part” refers in particular to joints and/or extremities, such as, for example, knees, ankles, shoulders, elbows or wrists. In the context of embodiments of the present invention, the term “body part” in particular refers to body areas, body regions and/or body parts of a patient that are located in multiple places and in particular at different locations on the patient's body.

The position of the body part can in particular comprise information characterizing the actual position of the body part. The position of the body part can substantially correspond to the position of the local coil. In particular, the position of the body part can be ascertained via an (image) analysis of the normalization scan. The actual position can in particular characterize the position of the local coil and/or of the body part at a specific time point. The position of the body part within the magnetic resonance apparatus (in particular with the patient in the examination position for the magnetic resonance examination) can preferably be ascertained immediately before the magnetic resonance examination. In particular, the position of the body part can characterize the positioning (actual position) of the body part in three spatial directions. Preferably, the position of the body part can describe the (ideally actual) spatial location of the actual position of the body part and/or local coil.

Preferably, the position of the body part can comprise a three-dimensional and/or two-dimensional description (in x and y) of a measurement point based on a reference object, in particular a reference point, via a coordinate system. In particular, the position of the body part can comprise an actual position of the local coil and/or of the body part with respect to a coordinate system and/or reference point. The reference point (e.g., zero point of the coordinate system) can preferably be an isocenter of the magnetic resonance apparatus and/or a reference point of the patient table (for example, a table end and/or table center). The reference point can preferably be a fixed point of the magnetic resonance apparatus. The position of the body part can in particular be a distance and/or path length. In particular, the position of the body part can be a height specification and/or a length value of a path in the x, y and/or z direction of a coordinate system. In particular, the position of the body part can be transferred/output to a system interface and/or user interface.

An alignment scan, also called a scout view, pre-scan or scout scan, is in particular a preparatory magnetic resonance scan with alignment scan parameters. The alignment scan can be used to ensure the positioning of a target region for a magnetic resonance scan. In particular, the alignment scan is performed before the magnetic resonance scan. The alignment scan comprises orthogonal (rectangular) images of the target region, body part and/or anatomical region of interest. In particular, this can ensure that slice images and/or slice positions for subsequent magnetic resonance scans are aligned precisely and correctly. In particular, the alignment scan is necessary when acquiring and/or positioning specific body parts, such as, for example, an elbow, in order to ensure that the imaging is suitable for diagnostic use.

Alignment scan parameters are in particular settings, values, information and/or technical data that enable the alignment scan to be controlled. The alignment scan parameters comprise, for example, the scan parameters for the magnetic resonance apparatus used to acquire the alignment scan.

An alignment process, also known as auto-align, can be performed prior to a magnetic resonance scan based on the image data from the alignment scan. The alignment process can automatically determine the slice positioning for the magnetic resonance scan. For example, the alignment process can comprise multiple iterations of adapting the slice positioning.

A normalization scan, also known as pre-scan normalize or normalize scan, is image acquisition for calibrating signal parameters of a local coil of the magnetic resonance apparatus. In particular, the normalization scan can be executed prior to a magnetic resonance scan. Calibration typically results in correct normalization of the magnetic resonance image data and compensation for signal inhomogeneities when acquiring a magnetic resonance scan. Calibration can in particular comprise determining data for image correction. In particular, the data determined from the normalization scan can be used to correct a magnetic resonance scan of the patient's body part.

The normalization scan typically comprises the entire patient-receiving region, also field of view of the magnet bore, of the magnetic resonance apparatus. Typically, the normalization scan is acquired in low resolution from the magnetic resonance scan and/or an alignment scan. The parameters for the normalization scan are in particular predetermined. The parameters for the normalization scan are in particular set to ensure homogeneous and consistent acquisition. For this purpose, the signal intensity can be adapted and/or varied across the patient-receiving region. Differences in the signal strength caused by the local coil can be minimized by calibrating the local coil and/or magnetic resonance apparatus.

The alignment scan parameters for the alignment scan based on the determined position of the body part can in particular be ascertained via a computing unit. The information about the position of the body part can be analyzed via an algorithm, function and/or computer program product executed in particular on the computing unit. For example, the alignment scan parameters can be determined from the coordinates and spatial dimensions of the body part. An algorithm and/or function can in particular determine the location and alignment of the body part from the captured position of the body part. In particular, the alignment scan parameters can be derived from the determined location and alignment of the body part. An algorithm and/or function can, for example, calculate the alignment scan parameters, such as the angles of the coils, the intensity of the magnetic field and the duration of the scan, from coordinates of the body part. In other words, the alignment scan parameters can be determined directly from the information and/or data from the normalization scan without further image acquisition, such as, for example, a first alignment scan.

The alignment scan parameters can preferably be ascertained via a neural network. The neural network is embodied to analyze the position of the body part and to output alignment scan parameters. The neural network can have been trained using previous magnetic resonance scans, alignment scans and/or normalization scans for different body parts.

The provision of the alignment scan parameters can in particular comprise provision to an operator of the magnetic resonance apparatus, as well as displaying, transmitting, storing or processing the alignment scan parameters. The alignment scan parameters can, for example, be displayed to an operator via a display unit of the magnetic resonance apparatus. The alignment scan parameters can, for example, be stored in a database, a computing unit and/or control unit, transmitted thereto and/or further processed thereby.

In particular, the provision of the alignment scan parameters can comprise generating a depiction of the alignment scan parameters for display to an operator in a user interface. The depiction optionally comprises one or more response options. The generation of the depiction optionally comprises creating at least one response option from the alignment scan parameters. The depiction can be provided to the operator in the user interface. The method can comprise receiving, optionally via the user interface, user input aimed at selecting a response option.

Advantageously, the proposed method can reduce the time required for alignment scans in magnetic resonance examinations. Herein, it is particularly advantageous to ascertain alignment scan parameters from the normalization scan, since the normalization scan has to be performed to calibrate the local coil. Hence, the data from the normalization scan can advantageously be used to determine alignment scan parameters. In other words, available data from the normalization scan required for calibration can be used more efficiently and/or for (further) parameterization of the alignment scan. Typically, a plurality of alignment scans is to be acquired iteratively. Typically, the required alignment scan parameters are determined iteratively from the plurality of alignment scans. The number of these alignment scans can be advantageously reduced by the proposed method. In addition, the amount of data or the information to be stored from alignment scans can be reduced. In addition, the proposed method is efficient and can be implemented with low computational effort. Overall, the proposed method advantageously enables the examination time to be reduced.

According to a preferred aspect of one or more example embodiments of the present invention, the normalization scan comprises capturing three-dimensional data from the patient-receiving region of the magnetic resonance apparatus with predefined parameters.

In particular, the normalization scan can be performed in accordance with a normalization scan protocol.

The normalization scan protocol can comprise a plurality of predefined parameters. In particular, a plurality of parameters can determine a measurement protocol and/or a measurement sequence for a normalization scan. A parameter for the normalization scan can in particular comprise information for controlling a magnetic resonance apparatus, in particular a protocol for performing a normalization scan. For example, the predefined parameter can comprise slice spacing, slice number, slice thickness, acquisition time and/or acquisition resolution. In other words, the parameters can in particular comprise information about the type of imaging, parameters, values, setting information and/or a protocol for the magnetic resonance apparatus.

The three-dimensional data from the normalization scan can in particular characterize acquisition of the patient-receiving region and/or the magnet bore, in particular in three spatial directions. Preferably, the position of the body part can describe the (ideally actual) spatial location of the actual position of the body part and/or local coil. The three-dimensional data from the normalization scan can comprise a three-dimensional description (in x, y and z) of the measurement point starting from a reference object, in particular a reference point, via a coordinate system. The reference point (e.g., zero point of the coordinate system) can preferably be an isocenter of the magnetic resonance apparatus and/or a fixed point of the magnetic resonance apparatus. In particular, the three-dimensional data from the normalization scan can be transferred/output to a system interface and/or user interface.

The patient-receiving region is in particular the region of the magnetic resonance apparatus in which a patient can be positioned for a magnetic resonance examination. In particular, the patient-receiving region can be the maximum acquisition region of the magnetic resonance apparatus. In particular, the acquisition region and/or the parameters of the normalization scan can be predetermined by the patient-receiving region.

Advantageously, three-dimensional data from the patient-receiving region of the magnetic resonance apparatus captured by a normalization scan with predefined parameters enables efficient determination of the position of the body part.

According to a preferred aspect of one or more example embodiments of the present invention, determining the position of the body part comprises ascertaining data values of the normalization scan that lie within a predetermined threshold range. In addition, determining the position of the body part comprises ascertaining a region of the position of the body part based on the ascertained data values.

The data values can in particular be intensity values of the normalization scan. In particular, ascertaining the data values that lie within a predetermined threshold range can comprise dividing the scan information ascertained by the normalization scan into two or more, in particular three, spatial directions. A threshold range can be predetermined for each spatial direction. Alternatively and/or additionally, a general threshold range can be defined. Ascertaining the data values can in particular comprise determining all points arranged between two threshold values, above a threshold value and/or below a threshold value in a data space. In particular, a predetermined data value, for example “1”, can be inserted and/or stored for all data values of the normalization scan arranged between two threshold values, above a threshold value and/or below a threshold value in a data space. In particular, a further predetermined data value, for example “0”, can be inserted and/or stored for all data values of the normalization scan arranged outside a threshold range, above a threshold value and/or below a threshold value in a data space.

In particular, a region of the position of the body part can be ascertained based on the determination of the data values within the threshold range. For example, all data values of the normalization scan corresponding to a data value “1” can characterize the region of the position of the body part.

Advantageously, ascertaining a region of the position of the body part via a threshold value check enables the position of the body part to be ascertained from the data from the normalization scan in a reliable, simple and computationally efficient manner.

According to a preferred aspect of one or more example embodiments of the present invention, determining the position of the body part comprises ascertaining intensity values in three spatial directions of the normalization scan. In addition, determining the position of the body part comprises ascertaining at least one intensity maximum. In addition, determining the position of the body part comprises capturing a point of the position of the body part based on the intensity maximum.

In particular, a point can be determined using the data from the normalization scan by capturing intensity values along the three Cartesian axes (x, y and z). The intensity values are in particular numerical measured values representing the signal strength at a point in space. The intensity values can in particular be analyzed by a computing unit in order in particular to identify at least one local maximum of the intensity values. An intensity maximum in particular represents the point of highest and/or greatest signal strength. Localization of these intensity maxima enables the position of the body part within the magnetic resonance imaging scanner to be determined.

One advantage of determining the position of the body part via an intensity maximum is precise localization and identification of the body part. Capturing intensity values and intensity maxima, in particular in three spatial directions, enables the position of the body part to be determined efficiently.

According to a preferred aspect of one or more example embodiments of the present invention, ascertaining alignment scan parameters comprises positioning an alignment acquisition region based on the determined position of the body part.

According to a preferred aspect of one or more example embodiments of the present invention, the alignment acquisition region is a predetermined region based on the body part.

In particular, the alignment acquisition region can be a predefined region based on the body part. For example, a predetermined alignment acquisition region can be a body part defined as a knee. The alignment acquisition region can also be referred to as a scout acquisition region or scout box. The alignment acquisition region can in particular be predetermined in terms of shape, extent, dimensions and/or further parameters. Alternatively and/or additionally, the alignment acquisition region can be dynamically adaptable, in particular based on the body part and/or the position of the body part.

The positioning of the alignment acquisition region can in particular comprise so-called fitting of the alignment acquisition region to the specific position of the body part. Fitting can refer to iterative positioning of an alignment acquisition region, in particular by minimizing distance values from points of the alignment acquisition region (for example corner points and/or the center point) and points of the position of the body part (for example, the center point).

In particular, ascertaining the alignment scan parameters can comprise selecting parameters and/or an alignment scan protocol from predefined parameters and/or protocols based on the position of the body part.

Positioning the alignment acquisition region can advantageously enable visualization of the parameterization of the alignment scan. This can lead to more efficient performance of the alignment scan, since relevant regions of the body part are captured in a predefined and/or predetermined manner. In addition, the alignment scan results can be improved by an alignment acquisition region defined for each body part in each case. Dynamic adaptability of the alignment acquisition region can enable individual differences and/or specific requirements of the body part to be taken into account, thereby enabling the flexibility and applicability of the method to be improved. Overall, an optimized alignment scan workflow can be achieved for an operator, in particular through the simplified determination of an alignment acquisition region.

According to a further aspect of one or more example embodiments of the present invention, the method additionally optionally comprises capturing user input. The user input can be used to effect a change in the positioning of the alignment acquisition region.

The user input can, for example, be captured via an output unit, in particular via a user interface comprised by the output unit. In particular, the user input can be dependent on the determined position of the body part. For example, the specific position of the body part can be provided to the operator of the magnetic resonance apparatus in conjunction with a proposed positioning of the alignment acquisition region. In addition, response options for user input can be provided to the operator.

In particular, ascertaining alignment scan parameters can comprise generating a depiction of the alignment acquisition region to be displayed to an operator in a user interface. The depiction optionally comprises one or more response options. The generation of the depiction optionally comprises creating at least one response option from the position of the body part. The depiction can be provided to the operator in the user interface. The method can comprise receiving, optionally via the user interface, user input aimed at selecting a response option.

Advantageously, capturing user input can enable a simple correction of the alignment acquisition region.

According to a preferred aspect of one or more example embodiments of the present invention, the alignment acquisition region comprises a predefined buffer region depending on the body part.

A buffer region can in particular be an additional region positioned around the captured body part. The buffer region can be embodied by a corresponding extension around the body part to ensure that the body part is completely acquired by the alignment scan, for example even in the case of possible movements on the part of the patient. The buffer region can in particular be a predefined region around the body part to be examined, depending on the body part. In particular, the buffer region can be defined by a distance value. A distance value of, for example, 5 cm can determine a region with a width of at least 5 cm around the body part.

The additional region of the buffer region around the body part to be examined advantageously enables it to be ensured that, even with slight movements on the part of the patient during the magnetic resonance scan, the relevant signals are captured and image quality is not impaired.

According to a preferred aspect of one or more example embodiments of the present invention, determining the position of the body part based on the normalization scan comprises determining a body side depending on the patient's position.

Typically, in magnetic resonance examinations, a differentiation is also made between two positions of the patient: head-first or feet-first. When the patient is in the head-first position, the position of the head is clearly defined, primarily by the use of a head coil. In the head-first position, the head of the patient is moved first into the patient-receiving region. When the patient is in the feet-first position, the patient's feet are moved first into the patient-receiving region.

The body side in particular refers to the lateral orientation of a body part of the human body in relation to the midline. Basically, the body can be divided into two symmetrical halves, a right body side and a left body side. The right body side comprises all body parts that are located to the right of the midline of the body, depending on the position of the patient, such as, for example, the right arm, the right hip, the right elbow and/or the right ankle. Similarly, the left body side comprises all body parts to the left of the midline, including the left arm, the left hip, the left elbow and the left ankle.

In other words, the body side refers to the lateral orientation of body parts of the human body relative to the midline.

Advantageously, the body side can be determined for bilateral body parts of the patient. This distinction can enable accurate and reliable verification of the location and position of the local coil on the body part.

According to a preferred aspect of one or more example embodiments of the present invention, the method comprises capturing an alignment scan based on the alignment scan parameters. In addition, the method comprises ascertaining slice-planning information for a magnetic resonance scan based on the alignment scan. In addition, the method comprises capturing a magnetic resonance scan of the patient's body part.

A magnetic resonance scan comprises medical image data from the body region of the patient. The medical image data can in particular comprise data and/or information generated during a medical imaging examination via the magnetic resonance apparatus, in particular based on an imaging protocol. The medical image data can, for example, contain information about the intensity and/or phase of magnetic resonance or X-ray radiation that is, for example, reflected, absorbed or emitted by a patient. The medical image data can in particular comprise data in various data formats, for example raw data, K-space data or image-space data. The medical image data can in particular be used as input data for the reconstruction of medical images. The medical image data can in particular comprise information, in particular reconstructed image data, about the anatomical and/or functional properties of the patient being examined. The medical image data can in particular comprise signals captured during a medical imaging examination.

Slice-planning information can in particular comprise information for planning a magnetic resonance scan of the patient's body part. For example, slice-planning information can comprise information for controlling the magnetic resonance apparatus. Slice-planning information can, for example, comprise imaging parameters, in particular a measurement protocol for performing a magnetic resonance scan. For example, slice-planning information (also imaging parameters) can comprise slice spacing, slice number, slice thickness, a turbo factor and/or a flip angle. In other words, the slice-planning information can in particular comprise information about a type of examination and/or imaging apparatus, parameters, values, setting information and/or a protocol for the medical imaging examination.

Advantageously, the optimized alignment scan enables precise and efficient determination of the slice-planning information. This can enable calibration and/or adjustment of the magnetic resonance apparatus. This can improve the image quality of the magnetic resonance scans and/or reduce image artifacts and inhomogeneities. In addition, the method can advantageously lead to more efficient use of the magnetic resonance apparatus, shorter scan times and/or reduced need for manual adaptation by an operator.

One or more example embodiments of the present invention also propose a magnetic resonance apparatus for capturing a magnetic resonance scan of a patient's body part. The magnetic resonance apparatus is embodied to execute a method according to one of the above-described aspects.

In particular, a magnet unit of the magnetic resonance apparatus can comprise a main magnet, a gradient coil unit and a radio-frequency antenna unit. The main magnet of the magnet unit is preferably embodied to generate a homogeneous (strong, constant) main magnetic field with a defined and/or specific magnetic field strength, such as, for example, with a defined and/or specific magnetic field strength of 3 T or 1.5 T. The homogeneous main magnetic field is preferably arranged and/or located within a patient-receiving region of the magnetic resonance apparatus. The magnet unit typically surrounds the patient region (or patient-receiving region) embodied to receive a patient for a magnetic resonance examination. The gradient coil unit is preferably embodied to generate gradient fields that are used for position encoding during imaging. The radio-frequency antenna unit is preferably fixed within the magnet unit and designed and/or embodied to emit an excitation pulse. To capture the magnetic resonance signals, the magnetic resonance apparatus preferably has local radio-frequency coils arranged around the region of the patient to be examined.

The advantages of the proposed magnetic resonance apparatus substantially correspond to the advantages of the proposed method. Features, advantages or alternative embodiments/aspects of the method can likewise be transferred to the other claimed subject matter and vice versa.

According to a further aspect of one or more example embodiments of the present invention, the magnetic resonance apparatus comprises a computing unit.

The computing unit can be comprised by a system control unit of the magnetic resonance apparatus. The computing unit can in particular be embodied to control and/or regulate the magnetic resonance apparatus. The computing unit can in particular be embodied to execute a method according to one of the above-described aspects.

Furthermore, a computer program product is proposed, which comprises a program and can be loaded directly into a memory of a programmable system control unit of a magnetic resonance apparatus and has program instructions and/or program means, e.g., libraries and auxiliary functions, for executing a proposed method when the computer program product is executed in the system control unit of the magnetic resonance apparatus and/or local coil. Herein, the computer program product can comprise software with a source code that still has to be compiled and linked or only has to be interpreted, or executable software code that only needs to be loaded into the system control unit for execution.

The computer program product advantageously enables the proposed method to be executed quickly, identically repeatedly and robustly. The computer program product is preferably configured such that it can execute the proposed method steps via the system control unit. Herein, the system control unit in each case has the requisite conditions such as, for example, an appropriate random access memory, an appropriate graphics card or an appropriate logic unit, so that the respective method steps can be executed efficiently.

The computer program product is, for example, stored on a non-transitory computer-readable medium or on a network or server from where it can be loaded into the processor of a local system control unit that can be directly connected to the magnetic resonance apparatus and/or local coil or embodied as part of the magnetic resonance apparatus and/or local coil. Furthermore, control information of the computer program product can be stored on an electronically readable data carrier. The control information of the electronically readable data carrier can be designed such that it performs a proposed method when the data carrier is used in a system control unit of a magnetic resonance apparatus and/or local coil.

Examples of electronically readable data carriers are a DVD, a magnetic tape or a USB stick, on which electronically readable control information, in particular software, is stored. When this control information is read from the data carrier and stored in a system control unit of the medical imaging apparatus, all proposed embodiments of the above-described method can be performed.

### BRIEF DESCRIPTION OF THE DRAWINGS

Further advantages, features and details of the present invention emerge from the exemplary embodiments described below and from the drawings. Corresponding parts are designated by the same reference characters in all figures. Modifications mentioned in this context can in each case be combined with one another to form new embodiments.

The figures show:

FIG. 1 is a flow diagram of a method for providing alignment scan parameters for an alignment scan of a magnetic resonance examination of a patient's body part via a magnetic resonance apparatus according to a preferred embodiment with an optional method step for providing the consistency information.

FIG. 2 is a flow diagram of a method for providing alignment scan parameters comprising a plurality of optional method steps according to a preferred embodiment.

FIG. 3 is a data flow diagram of a method for providing alignment scan parameters according to an embodiment.

FIG. 4 is a schematic depiction of an embodiment of a magnetic resonance apparatus.

FIG. 5 is a schematic depiction of the position of the patient's body part with an alignment acquisition region based on the position of the body part.

FIG. 6 is a schematic depiction of a signal intensity distribution of a normalization scan.

FIG. 7 is a schematic depiction of a determination of a region of the position of the body part.

### DETAILED DESCRIPTION

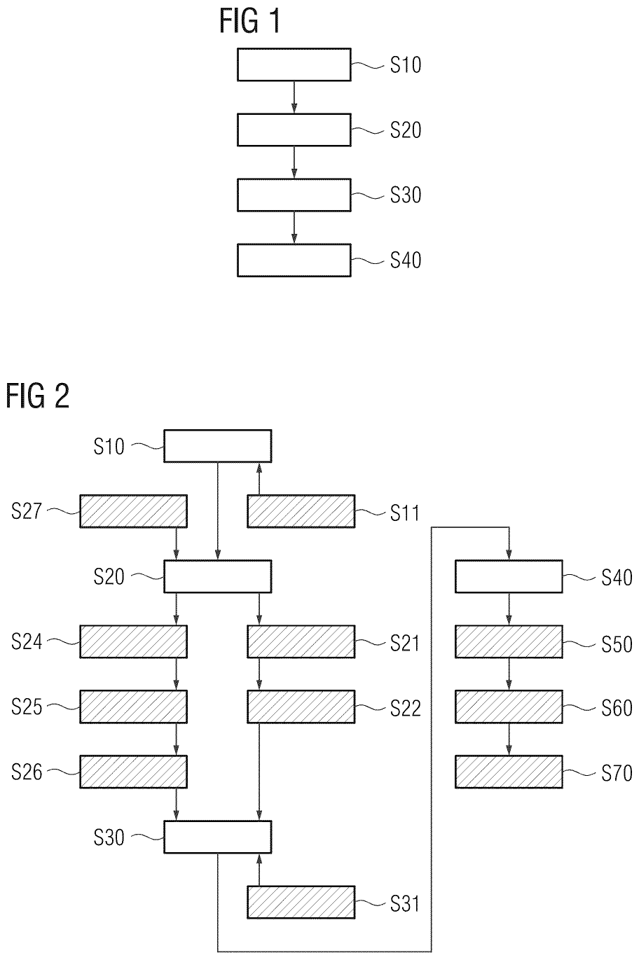

FIG. 1 shows a first exemplary embodiment of a method for providing alignment scan parameters for an alignment scan of a magnetic resonance examination of a patient's body part via a magnetic resonance apparatus. Here, FIG. 1 shows the main method steps of the method in an embodiment, while, in addition to the main steps of the method, FIG. 2 illustrates a plurality of optimal method steps and/or sub-steps of the method in an embodiment.

In a first method step of capturing S10 a normalization scan, measurement data for the normalization scan is captured via the magnetic resonance apparatus. The measurement data for the normalization scan can be provided to a computing unit. In particular, the normalization scan can be performed using a normalization scan protocol. Here, the normalization scan can correspond to image acquisition of the maximum image acquisition region of the magnetic resonance apparatus. The maximum image acquisition region can in particular correspond to the patient-receiving region and/or the magnet bore of the magnetic resonance apparatus. The normalization scan can in particular have a lower resolution, a short acquisition time and/or a larger acquisition region than the magnetic resonance scan and/or the alignment scan. These parameters and/or properties of the normalization scan can in particular be predetermined by a normalization scan protocol.

The capturing of the normalization scan in particular comprises capturing S11 three-dimensional data from the patient-receiving region of the magnetic resonance apparatus with predefined parameters. The measurement data for the normalization scan can comprise detailed position information for the local coil, in particular a three-dimensional spatial description (in x, y and z) of a measurement point and/or measurement region, in particular starting from a reference point. Preferably, the measurement data for the normalization scan can be depicted and provided via a coordinate system.

In a second method step S20, a position of the body part is determined based on the normalization scan. Positioning information can be ascertained from the data from the normalization scan. The position of the body part can comprise a three-dimensional spatial description (in x, y and z) of a point and/or region, in particular starting from a reference point. Preferably, the position of the body part can be depicted and provided as positioning information in a coordinate system.

In particular, the position of the body part can be determined by ascertaining S21 data values of the normalization scan that lie within a predetermined threshold range SW. The data values can in particular be intensity values (of a spatial direction). In particular, the threshold range SW can be predetermined by the body part to be examined. The threshold range SW can also be adapted by an operator and/or can be defined, in particular via a user interface. For example, the threshold range SW can be determined by a percentage value, for example 70 to 90 percent, preferably 80 percent, of an intensity value I of the normalization scan. As shown in FIG. 7, the threshold range SW can be determined by two limit values A, B. As depicted in FIG. 7, the region of the position of the body part KB can be determined by two values Pb1, Pb2.

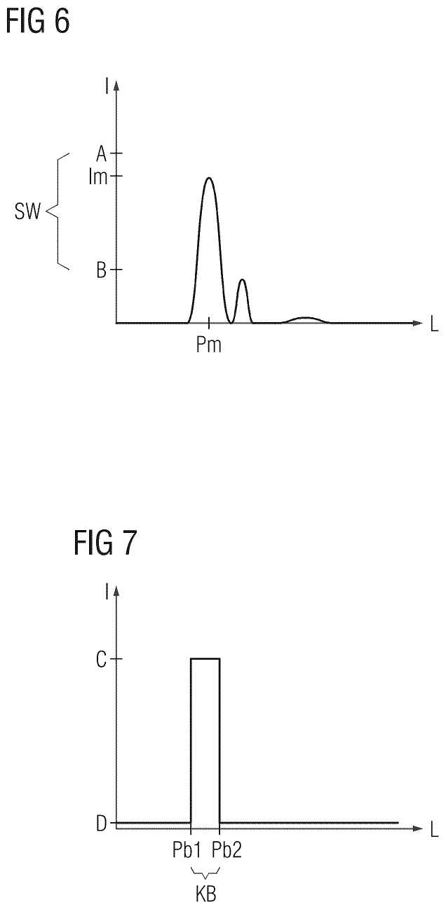

Ascertaining S21 data values of the normalization scan can in particular comprise checking the intensity values I of the normalization scan to determine whether they fall within or outside a threshold range SW. As illustrated in FIG. 6 and FIG. 7, a value C is set for the intensity value I if it falls within the threshold range SW. If the intensity value I falls outside the threshold range SW, a value D can be set.

The position of the body part can now be determined in a sub-step S22 by ascertaining a region of the position of the body part KB. As depicted in FIG. 7, the region of the position of the body part KB can be determined from the values Pb1, Pb2. Here, the values Pb1, Pb2 characterize the maximum or minimum position values L at which the intensity value is C.

In particular, the position of the body part can be determined by ascertaining S24 intensity values in three spatial directions of the normalization scan. The intensity values I (in three spatial directions) can be the signal strength of the magnetic resonance response of the patient's body, in particular the body part, recorded and/or acquired by the local coil. The spatial directions can comprise three directions, in particular vertical or parallel to the Earth's gravitational force, horizontal or orthogonal to the Earth's gravitational force and horizontal rotated 90° counterclockwise.

In an optimal sub-step S25 an intensity maximum Im can be ascertained for each spatial direction. As depicted in FIG. 6 by way of example for one spatial direction, a plurality of intensity maxima can occur in one direction. In this case, the intensity maximum Im with the highest intensity value Im can preferably be determined.

In an optimal sub-step S26, a point of the position of the body part Pm can be determined in a spatial direction depending on the intensity maximum Im. The point of the position of the body part Pm can in particular be a center point, starting point and/or reference point of a region of the position of the body part KB. In particular, a region of the position of the body part KB can be determined from the points of the position of the body part in the three spatial directions Pm.

In an optimal sub-step S27, the normalization scan can be used to determine a body side, in particular for body parts occurring on both sides, depending on the patient's position. Typically, two positions are known for patients in magnetic resonance apparatuses: the head-first position and the foot-first position. The body side for an alignment scan and/or magnetic resonance scan can be determined depending on a predetermined position of the patient. For example, ascertaining the intensity values in a spatial direction makes it possible to determine whether the body part, in particular characterized by an intensity maximum, is arranged closer to a right and/or left side of an inner wall of the magnet bore of the magnetic resonance apparatus. In particular, the body side of the body part can be determined by distributing the intensity values in a spatial direction. For example, as depicted in FIG. 6, more than one intensity maximum can be ascertained during a normalization scan of an elbow. Determining the sequence of the intensity maxima enables the body side of the body part to be ascertained.

In a third method step S30 of ascertaining alignment scan parameters for an alignment scan based on the determined position of the body part, the alignment scan parameters are preferably determined. Here, for example, it is possible to apply adaptation functions and/or fitting methods to adapt the acquisition region of the alignment scan to the position of the body part. The alignment scan parameters can be ascertained by a computing unit embodied to analyze the position of the body part and determine an acquisition region therefrom.

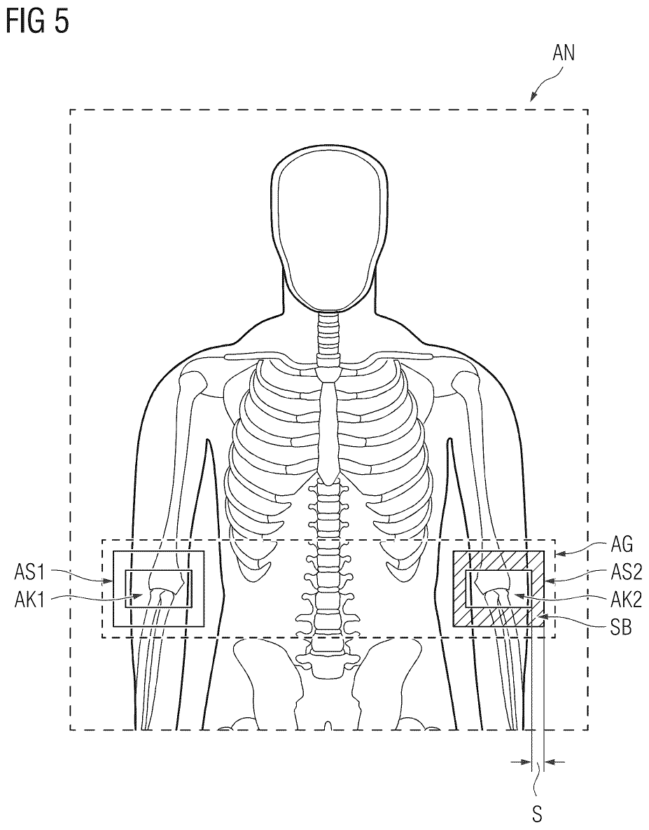

The optimal method step S31, the positioning of the alignment acquisition region AS1, AS2, is also illustrated in FIG. 5. The alignment acquisition region AS1, AS2 can preferably be a region predetermined with respect to the dimensions based on the body part AK1, AK2. In particular, the alignment acquisition region AS1, AS2 can comprise a predefined buffer region SB depending on the body part. The buffer region SB can in particular be determined via a distance value S. The alignment acquisition region AS1, AS2 can in particular be positioned on the basis of a region AK1, AK2 and/or point (not depicted) of the position of the body part. For example, the alignment acquisition region AS1 can be positioned based on the determined position of the body part AK1. In particular, the positioning S31 can be performed by minimizing distance values between a region AK1, AK2 and/or point of the position of the body part and the circumferential values of the alignment acquisition region AS1, AS2.

In a fourth method step S40, the alignment scan parameters are provided. These alignment scan parameters can be used to ensure that the acquisition region of the alignment scan is correctly positioned and alignment image data for a predetermined body part is captured. The alignment scan parameters can, for example, be provided to a control unit of the magnetic resonance apparatus for controlling an alignment scan. In particular, the alignment scan parameters can be provided for use on any computing unit, in particular a magnetic resonance apparatus.

Optimally, the alignment scan parameters can in particular be output to an operator. The provision of the alignment scan parameters can comprise outputting the alignment scan parameters. The alignment scan parameters can in particular be output in the form of a protocol. In particular, the alignment scan parameters can be output via a user interface and/or output unit, in particular on the magnetic resonance apparatus.

In an optional method step S50, an alignment scan is captured based on the alignment scan parameters. The alignment scan parameters can be used to set the magnetic resonance apparatus for an alignment scan of the patient's body part. Capturing the alignment scan S50 can comprise capturing alignment scan image data via the magnetic resonance apparatus.

In an optional method step S60, slice-planning information for a magnetic resonance scan is ascertained based on the alignment scan. The slice-planning information can be ascertained S60 by a computing unit. Ascertaining slice-planning information S60 in particular comprises determining slice positions and slice orientations based on the captured alignment scan image data. The slice-planning information can then be used to define the slice parameters for a magnetic resonance scan.

Capturing the magnetic resonance scans S70 in particular comprises performing a medical imaging examination using the magnetic resonance apparatus. Capturing the medical image data can also comprise sub-steps necessary for preparing for a medical imaging examination via the magnetic resonance apparatus. During the medical imaging examination, medical image data from the patient's body part is acquired according to an imaging protocol.

The method steps of capturing S10 measurement information MI and ascertaining positioning information PI can also be executed simultaneously or in reverse order. In addition, the method steps S10, S20, S30 (and S40) can be executed multiple times and/or iteratively. In addition, the method steps S10, S20, S30 (and S40) can in particular be executed before, but also after and during an imaging examination (or a plurality of imaging examinations) using the magnetic resonance apparatus 10.

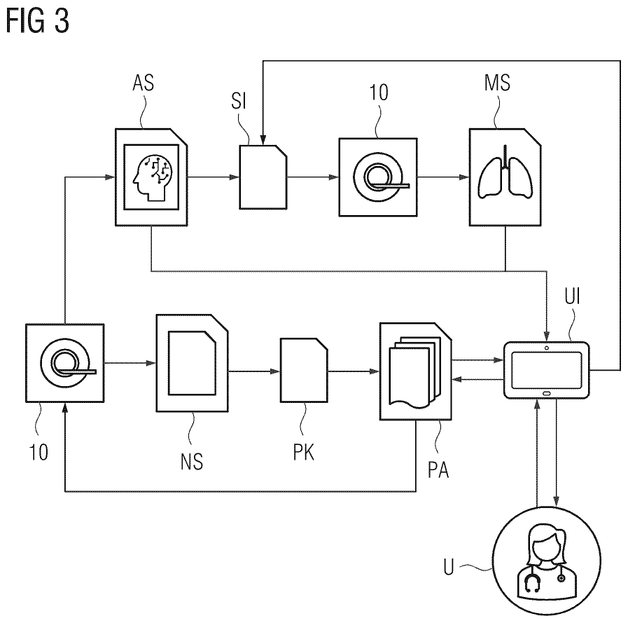

FIG. 3 illustrates the data flow of a method for providing alignment scan parameters PA for an alignment scan AS for a magnetic resonance scan MS of a patient's body part via a magnetic resonance apparatus 10 according to one embodiment.

A normalization scan NS is used to determine the position PK of the patient's body part. The position PK of the patient's body part comprises position information about a patient's body region (that is to be examined). The position PK of the patient's body part can comprise a position of a local coil arranged on a body region of the patient in the magnetic resonance apparatus 10. The position PK of the patient's body part can be determined from (the data from) the normalization scan NS via a computing unit (not depicted).

The alignment scan parameters PA for an alignment scan AS are ascertained from the position PK of the patient's body part using the magnetic resonance apparatus 10. The alignment scan parameters PA can be ascertained via a computing unit (not depicted). The alignment scan parameters PA can be queried, displayed and/or modified via a user interface UI by an operator U. The alignment scan parameters PA can depict the starting information for setting the parameters of the magnetic resonance apparatus 10.

The alignment scan parameters PA are used to perform an alignment scan AS by the magnetic resonance apparatus 10. The data from the alignment scan AS can be used to ascertain the slice-planning information SI for a magnetic resonance scan MS. The slice-planning information SI can be ascertained via a computing unit (not depicted). The slice-planning information SI can depict the output data for the magnetic resonance scan MS by the magnetic resonance apparatus 10. The data from the alignment scan AS can be displayed and/or illustrated to an operator U via the user interface UI. The operator can modify the slice-planning information SI via an input.

The operator U of the magnetic resonance apparatus 10 can be enabled to enter user input via the user interface UI. The user input can be captured from the user interface UI and processed. Depending on the user input, the user interface UI can transmit information to a computing unit (not depicted), in particular of the magnetic resonance apparatus 10. For example, the slice-planning information SI, the alignment scan parameters PA and/or other information can be adapted by the user interface UI via the user input. In other words, the alignment scan AS and/or magnetic resonance scan MS can be adapted and/or controlled via the user interface UI via user input.

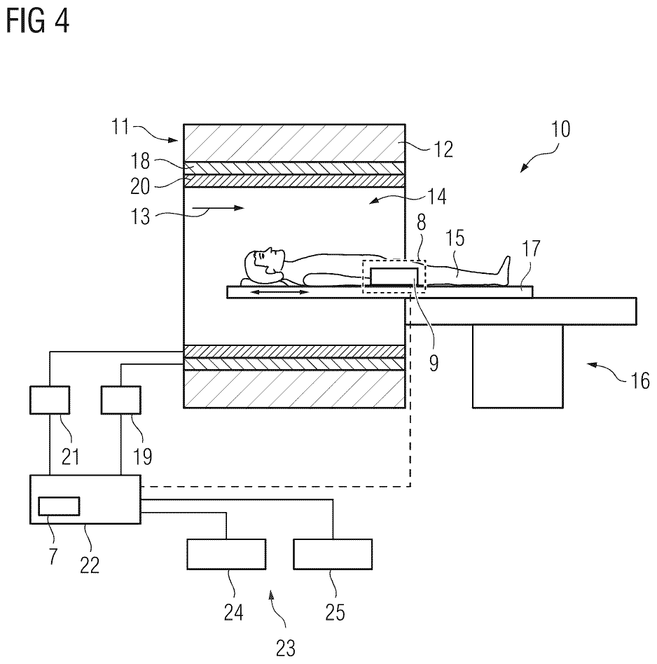

FIG. 4 is a schematic depiction of a magnetic resonance apparatus 10. The magnetic resonance apparatus 10 comprises a magnet unit 11 having a main magnet 12 for generating a strong and in particular temporally constant main magnetic field 13. In addition, the magnetic resonance apparatus 10 has a magnet bore 14 for receiving a patient 15, i.e., the magnet bore 14 can be regarded as a patient-receiving region. The magnet bore 14 is embodied in the form of a cylinder with a central axis z in the z-direction and is surrounded by the magnet unit 11 in a circumferential direction. The magnet bore 14 is bounded by an inner wall W of the magnet unit 11. The patient 15 can be pushed into the magnet bore 14 via a patient positioning apparatus 16 of the magnetic resonance apparatus 10. For this purpose, the patient positioning apparatus 16 has a patient table 17 designed to be movable within the magnet bore 14.

The magnet unit 11 further has a gradient coil unit 18 with at least one gradient coil for generating magnetic field gradients that are used for spatial encoding during imaging. The gradient coil unit 18 is controlled via a gradient control unit 19 of the magnetic resonance apparatus 10. The magnet unit 11 further comprises a radio-frequency antenna unit 20 with at least one transmitting antenna that, in the present exemplary embodiment, is embodied as a whole-body coil permanently integrated in the magnetic resonance apparatus 10. The radio-frequency antenna unit 20 is controlled by a radio-frequency control unit 21 of the magnetic resonance apparatus 10 and irradiates radio-frequency magnetic resonance sequences, in particular RF transmission pulses, into a patient-receiving region that is substantially formed by the region of the magnet bore 14 of the magnetic resonance apparatus 10. As a result, the main magnetic field 13 generated by the main magnet 12 causes an excitation of atomic nuclei in that nuclear spins are deflected from their rest position. Magnetic resonance signals are generated by relaxation of the excited atomic nuclei. The radio-frequency antenna unit 20 can basically be embodied to receive the magnetic resonance signals.

In addition to the radio-frequency antenna unit 20 and/or comprised thereby, the magnetic resonance apparatus can comprise a local coil 9. Herein, the local coil 9 can be embodied similarly to the radio-frequency antenna unit 20. However, in contrast to the radio-frequency antenna unit 20, the local coil 9 can be positioned directly on the patient 15 around a body part of the patient 15.

Therefore, the magnet unit 11 can also comprise at least one local coil with a receiving antenna, which, in the present exemplary embodiment, is positioned in the magnet bore 14 of the magnetic resonance apparatus 10 on an arm of the patient 15, in particular the elbow. The local coil 9 can in particular receive radio-frequency magnetic resonance sequences, in particular RF transmission pulses from a patient-receiving region. The patient-receiving region is substantially formed by the region of the magnet bore 14 of the magnetic resonance apparatus 10. In principle, the local coil 9 can be embodied to receive the magnetic resonance signals or the relaxation of the excited atomic nuclei of the body part of the patient 15.

To control the main magnet 12, the gradient control unit 19 and to control the radio-frequency control unit 21, the magnetic resonance apparatus 10 has a system control unit 22. The system control unit 22 centrally controls the magnetic resonance apparatus 10, such as, for example, performing a predetermined imaging gradient echo sequence. In addition, the system control unit 22 comprises an evaluation unit (not shown in detail) for evaluating the magnetic resonance signals captured during the magnetic resonance examination. Moreover, the magnetic resonance apparatus 10 comprises a user interface 23 connected to the system control unit 22. Control information, such as, for example, imaging parameters and reconstructed magnetic resonance images can be displayed on a display unit 24, for example on at least one monitor, of the user interface 23 for a medical operator. Furthermore, the user interface 23 has an input unit 25 by which information and/or parameters can be input by the medical operator during a measurement process.

The magnetic resonance apparatus 10, in particular the system control unit 22, can also comprise a computing unit 7. The computing unit 7 can be embodied to ascertain the position of the body part. The computing unit 7 can be embodied to ascertain the alignment scan parameters. In addition to ascertaining the alignment scan parameters, ascertaining the body part can ensure that no other body part of the patient 15 than that intended is accidentally acquired with the magnetic resonance apparatus 10. The computing unit 7 can be connected to the local coil. The computing unit 7 can be embodied to determine the position of the local coil 9 in the magnet bore 14.

The computing unit 7 can determine the position of the body part based on the data from the normalization scan. The computing unit 7 can correspond to the system control unit and be connected to the user interface 23. The computing unit 7 can be fully integrated into the system control unit 22. The computing unit 7 can be embodied to check whether a limit value and/or limit range, in particular the intensity of a signal from the normalization scan, is undershot, exceeded and/or maintained. If the limit value is undershot and/or exceeded, the computing unit 7 can send information to the user interface 23 in order to inform the operator and/or receive user input.

In contrast to the case depicted, the computing unit 7 can also be arranged outside the magnetic resonance apparatus 10. The computing unit 7 can be connected to the system control unit 22 for signal transmission.

FIG. 5 is a schematic depiction of the position AK1, AK2 of the patient's body part and the alignment acquisition regions AS1, AS2. The position AK1, AK2 of the patient's body part, here by way of example the patient's elbows, are illustrated by regions around the body part. However, unlike the case depicted, the position AK1, AK2 of the body part can be determined by a point. The alignment acquisition region AS1 comprises a buffer region SB, which is defined via a distance value S.

Also shown is the acquisition region AN of the normalization scan. The acquisition region AN of the normalization scan typically corresponds to the patient-receiving region or the region within the magnet bore of the magnetic resonance apparatus. Also illustrated is the acquisition region AG of the alignment scan without a determination of the position of the body part based on the normalization scan. Comparing the acquisition regions AG and AS1, AS2 or subtracting the area of the acquisition region AS1, AS2 from the acquisition region AG illustrates an advantage of the method, in particular the reduced acquisition region for an alignment scan.

FIG. 6 and FIG. 7 are schematic depictions of a signal intensity distribution of a normalization scan and a determination of a region KB of the position of the body part by determining data values of the normalization scan that lie within a predetermined threshold range SW. The signal intensity distribution is illustrated by plotting the intensity values I against position values L. Here, the position values L can, for example, characterize the position of the intensity values I via a distance to a reference point.

The threshold range SW is determined by two limit values A, B. The region of the position of the body part KB is determined by two values Pb1, Pb2. The value C can be the (specified) intensity value of the region of the position of the body part KB. The value D can be the intensity value for position values L outside the position of the body part. The region of the position of the body part KB can be determined by the values Pb1, Pb2. The values Pb1, Pb2 characterize the maximum, or minimum position values L at which the intensity value is C. The point of the position of the body part Pm characterizes an intensity maximum Im in a spatial direction.

Reference is made once again to the fact that the methods described in detail above and the magnetic resonance apparatus depicted are merely exemplary embodiments which can be varied by the person skilled in the art in various ways without departing from the scope of the present invention. Furthermore, the use of the indefinite article “a” or “an” does not preclude the possibility that the features in question may also be present on a multiple basis. Likewise, the term “unit” does not preclude the possibility that the components in question may consist of a plurality of interacting subcomponents, which may also be spatially distributed. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternatively, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” or “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A method for providing alignment scan parameters for an alignment scan of a magnetic resonance examination of a body part of a patient via a magnetic resonance apparatus having a local coil, the method comprising:
capturing a normalization scan, wherein the normalization scan is used to calibrate the magnetic resonance apparatus;
determining a position of the body part based on the normalization scan, wherein the local coil is positioned in a patient-receiving region of the magnetic resonance apparatus around the body part;
ascertaining alignment scan parameters for an alignment scan based on the position of the body part; and
providing the alignment scan parameters.

2. The method as claimed in claim 1, wherein the normalization scan comprises:
capturing three-dimensional data from the patient-receiving region of the magnetic resonance apparatus with defined parameters.

3. The method as claimed in claim 1, wherein determining the position of the body part comprises:
ascertaining data values of the normalization scan that lie within a threshold range; and
ascertaining a region of the position of the body part based on the data values.

4. The method as claimed in claim 1, wherein determining the position of the body part comprises:
ascertaining intensity values in three spatial directions of the normalization scan;
ascertaining at least one intensity maximum; and
capturing a point of the position of the body part based on the at least one intensity maximum.

5. The method as claimed in claim 1, wherein ascertaining the alignment scan parameters comprises:
positioning an alignment acquisition region based on the position of the body part.

6. The method as claimed in claim 5, wherein the alignment acquisition region is a region based on the body part.

7. The method as claimed in claim 5, wherein the alignment acquisition region includes a buffer region depending on the body part.

8. The method as claimed in claim 1, wherein determining the position of the body part based on the normalization scan comprises:
determining a body side based on a position of the patient.

9. The method as claimed in claim 1, further comprising:
capturing an alignment scan based on the alignment scan parameters;
ascertaining slice-planning information for a magnetic resonance scan based on the alignment scan; and
capturing a magnetic resonance scan of the body part.

10. A magnetic resonance apparatus to capture a magnetic resonance scan of a body part, the magnetic resonance apparatus configured to perform the method as claimed in claim 1.

11. The method as claimed in claim 1, wherein the normalization scan is used to calibrate the local coil of the magnetic resonance apparatus.

12. The method as claimed in claim 2, wherein the three-dimensional data is captured according to a normalization scan protocol.

13. The method as claimed in claim 2, wherein determining the position of the body part comprises:
ascertaining data values of the normalization scan that lie within a threshold range; and
ascertaining a region of the position of the body part based on the data values.

14. The method as claimed in claim 13, wherein determining the position of the body part comprises:
ascertaining intensity values in three spatial directions of the normalization scan;
ascertaining at least one intensity maximum; and
capturing a point of the position of the body part based on the at least one intensity maximum.

15. The method as claimed in claim 2, wherein determining the position of the body part comprises:
ascertaining intensity values in three spatial directions of the normalization scan;
ascertaining at least one intensity maximum; and
capturing a point of the position of the body part based on the at least one intensity maximum.

16. The method as claimed in claim 3, wherein ascertaining the alignment scan parameters comprises:
positioning an alignment acquisition region based on the position of the body part.

17. The method as claimed in claim 4, wherein ascertaining the alignment scan parameters comprises:
positioning an alignment acquisition region based on the position of the body part.

18. The method as claimed in claim 6, wherein the alignment acquisition region includes a buffer region depending on the body part.

19. The method as claimed in claim 8, wherein the body side is determined for a body part occurring on both sides.

20. A non-transitory computer-readable medium storing computer-executable instructions that, when executed by at least one processor of a magnetic resonance apparatus, cause the magnetic resonance apparatus to perform the method as claimed in claim 1.
